SEQUENCE LISTING (2 sequences)

SEQ ID NO: 1 - DNA, 20 bp, synthetic construct
  source 1..20: mol_type = other DNA
aacatcgtgtcacccagcta

SEQ ID NO: 2 - DNA, 20 bp, synthetic construct
  source 1..20: mol_type = other DNA
cgtcacaactgctagaaggc

(19) United States
(12) Patent Application Publication
(10) Pub. No.: US 2026/0338802 A1
(43) Pub. Date: Oct. 29, 2026

(54) METHOD FOR THE DETECTION OF DEMENTIA

(71) Applicant: Belgian Volition SRL, Isnes (BE)

(72) Inventor: Jacob Vincent MICALLEF, Isnes (BE)

(21) Appl. No.: 19/106,422

(22) PCT Filed: Aug. 25, 2023
(86) PCT No.: PCT/EP2023/073347

§ 371 Date: Feb. 25, 2025

Related U.S. Application Data
(60) Provisional application No. 63/401,007, filed on Aug. 25, 2022.

(51) Int. Cl. G01N 33/68

(52) U.S. Cl. CPC G01N 33/6896; G01N 2440/18; G01N 2800/2821; G01N 2800/7095

(57) ABSTRACT

The invention relates to methods of detecting, diagnosing or monitoring an inflammatory condition of the central nervous system, in particular by detecting or measuring neutrophil extracellular traps, extracellular traps and/or cell free nucleosomes.

### FIELD OF THE INVENTION

The invention relates to the use of neutrophil extracellular traps, extracellular traps and/or cell free nucleosomes or a component thereof as a biomarker in a body fluid sample for diagnosing and/or monitoring of an inflammatory condition of the central nervous system, particularly but not exclusively dementia including Alzheimer's disease.

### BACKGROUND OF THE INVENTION

Dementia is a syndrome associated with an ongoing decline of brain functioning including memory loss and difficulties with thinking, problem-solving or language. There are many different causes of dementia, the most common being Alzheimer's disease (AD), accounting for 50 to 75% of dementia cases. AD is a progressive neurological disorder which is thought to be caused by the abnormal build-up of proteins in and around brain cells. One of these proteins is called amyloid, deposits of which form plaques around brain cells. The other is called tau, deposits of which form tangles within brain cells.

The symptoms of AD can be split into 3 main stages: early-stage AD (mild), middle-stage AD (moderate), and late-stage AD (severe). In the early stages of AD, the main symptom is memory lapses. At this stage, someone with AD may still function independently and perform their usual day-to-day activities such as driving and working. As the disease progresses into the middle stage, memory problems will worsen and the patient usually needs support to help with everyday living. In the later stages of AD, the symptoms become increasingly severe, and may include hallucinations and delusions. Patients at this stage may need full-time care and assistance with eating, moving and personal care.

Early, accurate diagnosis is beneficial for several reasons. While there is no cure for AD, there are several medicines available to treat the symptoms. Most medicines work best for patients in the early or middle stages of the disease, therefore beginning treatment early in the disease process may help preserve daily functioning. In addition, having an early diagnosis helps patients and their families plan for the future, take care of financial and legal matters, prepare living arrangements, and develop support networks.

There is currently no simple and reliable test for diagnosing AD. Diagnosis typically involves the use of cognitive tests (e.g., Mini-Mental State Exam (MMSE)), blood tests to check for other conditions, and brain scans. Cerebrospinal fluid (CSF) biomarkers are also increasingly used to support a diagnosis of AD. CSF amyloid beta (Aβ) 1-42, total tau (T-tau), and phosphorylated tau (P-tau) have utility in differentiating AD from controls and in predicting conversion from mild cognitive impairment (MCI) to AD. Consequently, these measures are included in clinical and research diagnostic criteria. However, the uptake of CSF biomarker analysis in the context of AD has been hampered by low rates of recommendation and the need to perform a lumbar puncture.

Methods for the detection, diagnosis and monitoring of other dementias and inflammatory conditions of the brain or central nervous system (CNS) have similar limitations. These include for example diagnosis, detection and monitoring of traumatic brain injury or chronic traumatic encephalopathy (CTE) or dementia pugilistica (also known as “punch drunk”). These conditions are reported to be effects of repeated head collisions, blows or concussions occurring in contact sports such as, without limitation, boxing, martial arts, American football, soccer, rugby and others.

There is therefore a need to develop tests, preferably non-invasive, for diagnosing and monitoring inflammatory conditions of the CNS, such as AD or other dementia, particularly in the early stages.

### SUMMARY OF THE INVENTION

According to an aspect of the invention there is provided a method of detecting, diagnosing or monitoring an inflammatory condition of the central nervous system (CNS) in a subject, comprising contacting a body fluid sample obtained from the subject with a binding agent to detect or measure the level of neutrophil extracellular traps (NETs), extracellular traps (ETs) or cell free nucleosomes or a component of NETs, ETs or cell free nucleosomes.

In one embodiment the inflammatory condition of the CNS is dementia.

In another embodiment the inflammatory condition of the CNS is AD.

In another embodiment the inflammatory condition of the CNS is Parkinson's disease.

In another embodiment the inflammatory condition of the CNS is traumatic brain injury or chronic traumatic encephalopathy.

According to another aspect of the invention there is provided a method of detecting, diagnosing or monitoring an inflammatory condition of the CNS in a subject, said method comprising obtaining a body fluid sample from the subject and detecting or measuring the level of NETs, ETs or cell free nucleosomes or a component of NETs, ETs or cell free nucleosomes to detect, diagnose or monitor the inflammatory condition of the CNS.

According to another aspect of the invention there is provided a method of detecting, diagnosing or monitoring an inflammatory condition of the CNS in a subject, said method comprising: (i) measuring or detecting levels of NETs, ETs or cell free nucleosomes or a component of NETs, ETs or cell free nucleosomes in a body fluid sample obtained from the subject, and (ii) comparing the levels determined in (i) to reference levels for NETs, ETs or cell free nucleosomes or the component thereof, respectively, to determine whether the subject is suffering with an inflammatory condition of the CNS or to monitor an inflammatory condition of the CNS in the subject.

According to another aspect of the invention there is provided a method for assessing the severity of an inflammatory condition of the CNS in a subject, comprising: (i) measuring levels of NETs, ETs or cell free nucleosomes or a component of NETs, ETs or cell free nucleosomes in a body fluid sample obtained from the subject, and (ii) comparing the levels determined in (i) to a reference level for NETs, ETs or cell free nucleosomes or the component thereof, respectively, to assess the severity of an inflammatory condition of the CNS in the subject.

According to another aspect of the invention there is provide a method of monitoring progression of an inflammatory condition of the CNS in a subject previously diagnosed with an inflammatory condition of the CNS, comprising (i) measuring levels of NETs, ETs or cell free nucleosomes or a component of NETs, ETs or cell free nucleosomes in a body fluid sample obtained from the subject, (ii) comparing the levels determined in (i) to a reference level for NETs, ETs or cell free nucleosomes or the component thereof, respectively, to monitor progression of an inflammatory condition of the CNS in the subject.

According to another aspect of the invention there is provided a method of treating an inflammatory condition of the CNS in a patient, comprising;
(i) detecting or measuring the level of NETs, ETs or cell free nucleosomes or a component of NETs, ETs or cell free nucleosomes in a body fluid sample obtained from the patient;
(ii) using the level of NETs, ETs or cell free nucleosomes or the component thereof measured in step (i) as indicative of the presence and/or severity of the inflammatory condition of the CNS in the patient; and
(iii) administering a treatment to the patient if they are determined to have an inflammatory condition of the CNS in step (ii).

According to another aspect of the invention there is provided a method of treatment for an inflammatory condition of the CNS in a subject comprising:
(i) obtaining a blood, serum or plasma sample from the subject;
(ii) measuring the level or amount of NETs, ETs or cell free nucleosomes or a component of NETs, ETs or cell free nucleosomes in the blood, serum or plasma sample;
(iii) using the level or amount of NETs, ETs or cell free nucleosomes of the component thereof measured in the blood, serum or plasma sample in step (ii) to identify the subject as being of high or low risk of an inflammatory condition of the CNS or progression of an inflammatory condition of the CNS;
(iv) obtaining a CSF sample from the subject, if the subject is identified as being of high risk of an inflammatory condition of the CNS or progression of an inflammatory condition of the CNS in step (iii);
(v) measuring the level or amount of NETs, ETs or cell free nucleosomes or the component thereof in the CSF sample; and
(vi) using the level or amount of NETs, ETs or cell free nucleosomes or the component thereof measured in the CSF sample in step (v) to identify the subject as being in need of treatment for an inflammatory condition of the CNS, and providing said treatment.

According to another aspect of the invention there is provided a method of detecting, diagnosing or monitoring an inflammatory condition of the CNS in a subject comprising:
(i) obtaining a blood, serum or plasma sample from the subject;
(ii) measuring the level or amount of NETs, ETs or cell free nucleosomes or a component of NETs, ETs or cell free nucleosomes in the blood, serum or plasma sample;
(iii) using the level or amount of NETs, ETs or cell free nucleosomes of the component thereof measured in the blood, serum or plasma sample in step (ii) to identify the subject as being of high or low risk of an inflammatory condition of the CNS or progression of an inflammatory condition of the CNS;
(iv) obtaining a CSF sample from the subject, if the subject is identified as being of high risk of an inflammatory condition of the CNS or progression of an inflammatory condition of the CNS in step (iii);
(v) measuring the level or amount of NETs, ETs or cell free nucleosomes or the component thereof in the CSF sample; and
(vi) using the level or amount of NETs, ETs or cell free nucleosomes or the component thereof measured in the CSF sample in step (v) to detect, diagnose or monitor an inflammatory condition of the CNS.

According to another aspect of the present invention there is provided use of the level of NETs, ETs or cell free nucleosomes or a component of NETs, ETs or cell free nucleosomes as a biomarker in a body fluid sample for detecting, diagnosing and/or monitoring an inflammatory condition of the CNS.

According to another aspect of the invention there is provided a kit comprising reagents to detect one or more nucleosome biomarkers selected from the group consisting of: nucleosomes containing the histone variant H3.1 (H3.1) and nucleosomes containing post translationally modified histone H3 citrullinated at the amino acid residue arginine 8 (H3R8cit), for use in diagnosing or monitoring an inflammatory condition of the CNS.

### BRIEF DESCRIPTION OF THE FIGURES

FIG. 1: Determining the level of nucleosomes comprising H3.1 in plasma samples obtained from Alzheimer patients with different dementia grades [Mild (n=6), Moderate (n=12) and Severe (n=7)] vs. age-matched healthy control patients (n=25).

FIG. 2: Determining the level of nucleosomes comprising H3R8Cit in plasma samples obtained from Alzheimer patients with different dementia grades [Mild (n=6), Moderate (n=12) and Severe (n=7)] vs. age-matched healthy control patients (n=25).

FIG. 3: Graph showing the median trend from the results obtained in FIGS. 1 and 2.

FIG. 4: Determining the level of Myeloperoxidase (MPO) using a commercially available kit in plasma samples obtained from Alzheimer patients with different dementia grades [Mild (n=6), Moderate (n=12) and Severe (n=7)] vs. age-matched healthy control patients (n=25).

FIG. 5: Graph showing the median trend from the results obtained in FIG. 4.

FIG. 6: Determining the level of cfDNA using a fluorescent DNA-binding dye method (Qubit DNA kit) in plasma samples obtained from Alzheimer patients with different dementia grades [Mild (n=6), Moderate (n=12) and Severe (n=7)] vs. age-matched healthy control patients (n=25).

FIG. 7: Determining the level of cfDNA using a qRT-PCR method in plasma samples obtained from Alzheimer patients with different dementia grades [Mild (n=6), Moderate (n=12) and Severe (n=7)] vs. age-matched healthy control patients (n=25).

FIG. 8: Graph showing the median trend from the results obtained in FIGS. 6 and 7.

FIG. 9: Determining the level of nucleosomes comprising H3.1 in plasma samples obtained from patients with traumatic brain injury (TBI) (n=6) vs. healthy control patients (n=269).

### DETAILED DESCRIPTION OF THE INVENTION

Recent studies provide evidence that inflammation plays an important role in dementia. Extended use of nonsteroidal anti-inflammatory drugs (NSAIDs) has been shown to result in a reduction in the risk for developing Alzheimer's disease (AD) (McGeer & McGeer, Sci. Aging Knowledge Environ. (2002) 2002 (29): re3). It has been suggested that in the early stages of AD, activation of microglia in the brain may serve a protective function by phagocytosing amyloid and recruiting further microglia. However, proinflammatory cytokines produced in response to amyloid deposition downregulate genes involved in amyloid clearance and promote amyloid accumulation, therefore contributing to neurodegeneration (Hickman et al., J. Neurosci. (2008) 28 (33): 8354-60).

Neutrophils have also been implicated in AD. Neutrophils are normally restricted from trafficking into the brain parenchyma and cerebrospinal fluid by the blood-brain barrier (BBB). However, neutrophils have been identified in the brains of patients with AD and in corresponding animal models and have been shown to be present in areas with amyloid deposits, where they release neutrophil extracellular traps (NETs) (Zenaro et al., Nat. Med. (2015) 21, 880-886). NETs are net-like structures originating from decondensed chromatin which are composed of DNA-histone complexes (i.e. nucleosomes) together with granule-derived antimicrobial peptides and enzymes such as neutrophil elastase (NE), cathepsin G, and myeloperoxidase (MPO). NET extrusion has also been detected in high concentrations in European AD patients' serum (Dong et al., Ann. Neurol. 2018 February; 83 (2): 387-405).

The present inventors have identified that elevated levels of NETs, ETs and/or cell free nucleosomes, or components of NETs, ETs or cell free nucleosomes may be used to diagnose and monitor and assist to in treating inflammatory associated conditions or disorders of the central nervous system, more particularly but not exclusively, inflammatory associated dementia conditions of the central nervous system (CNS) including AD and Parkinson's disease.

Therefore, according to the present invention, there is provided the use of the level of NETs, ETs or cell free nucleosomes or a component thereof as a biomarker in a body fluid sample for diagnosing and/or monitoring inflammatory associated dementia conditions of the central nervous system (CNS), including AD, Parkinson's disease and chronic traumatic encephalopathy (CTE).

The terms “extracellular traps” or “ETs” used throughout this document are intended to include any extracellular fragment in a body fluid pertaining to an ET or ETs or a component of an ET. It will be appreciated that references to “ETs” may be used herein as a shorthand reference for neutrophil extracellular traps or other extracellular traps, as in general terms several immune cells can release chromatin and granular proteins into extracellular space in response to the stimulation, forming ETs. The cells involved in the ET formation include neutrophils, macrophages, basophils, eosinophils, microglial cells and mast cells. ETs associated with neutrophils are generally referred to as “neutrophil extracellular traps” or “NETs”. The term “NETs” used throughout this document is intended to include any extracellular fragment in a body fluid pertaining to a NET or NETs or a component of a NET.

References to “nucleosome” may refer to “cell free nucleosome” when detected in body fluid samples. It will be appreciated that the term “cell free nucleosome” used throughout this document is intended to include any cell free chromatin fragment that includes one or more nucleosomes.

It will be understood that the cell free nucleosome may be detected by binding to a component thereof. The term “component thereof” as used herein refers to a part of the nucleosome, i.e. the whole nucleosome does not need to be detected. The component of the cell free nucleosomes may be selected from the group consisting of: a histone protein (i.e. histone H1, H2A, H2B, H3 or H4), a histone post-translational modification, such as citrullination, a histone variant or isoform, a protein bound to the nucleosome (i.e. a nucleosome-protein adduct), a DNA fragment associated with the nucleosome and/or a modified nucleotide associated with the nucleosome. For example, the component thereof may be histone (isoform) H3.1 or histone H1 or DNA.

Similarly, it will be appreciated that the NET or ET may be detected by binding to a component thereof, and that references to NET or ET may encompass the detection or monitoring of a component of the NET or ET. In this context the term “component thereof” as used herein refers to a part of the NET or ET, i.e. the whole NET or ET does not need to be detected. The component of the NETs or ETs may be, for example, myeloperoxidase (MPO) or DNA.

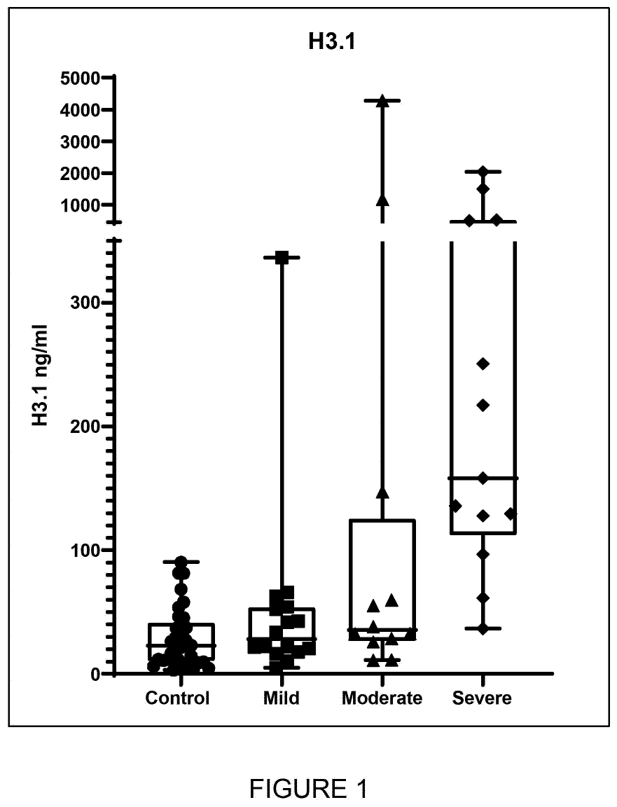

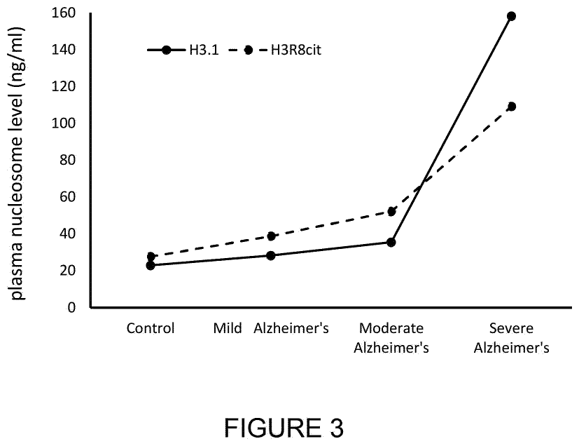

We measured the level of circulating cell free nucleosomes containing histone variant H3.1 in plasma samples obtained from subjects diagnosed with mild, moderate or severe AD. We observed disease stage dependent elevated levels in AD patients (FIGS. 1 and 3).

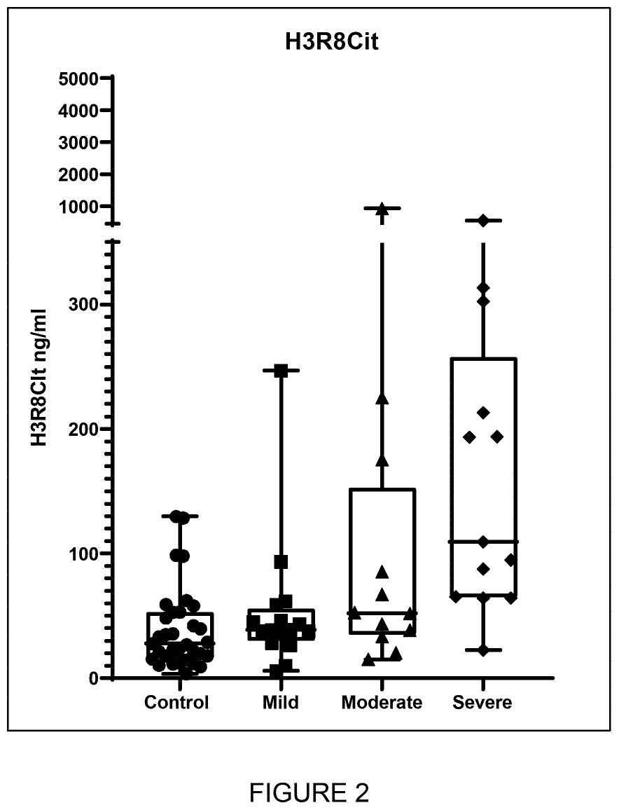

As chromatin released into the extracellular space by NETosis is reported to be citrullinated, we also measured the level of plasma cell free nucleosomes containing a citrullinated amino acid post translational modification (PTM) in the same patients. Nucleosomes may be citrullinated at a variety of amino acid positions. We selected to test for nucleosomes containing the PTM H3R8Cit (histone H3 citrullinated arginine at amino acid residue 8) in plasma samples obtained from subjects diagnosed with mild, moderate or severe AD. We observed disease stage dependent elevated levels of AD patients (FIGS. 2 and 3).

We have also measured elevated H3.1 and H3R8Cit levels in other diseases involving NETosis and observed that the ratio of H3R8Cit/H3.1 in these elevated nucleosome levels is usually <0.2 (i.e. the level of citrullinated nucleosomes is low). In the case of nucleosomes associated with inflammation of the CNS here, we observed ratios higher citrullination levels. The mean H3R8Cit/H3.1 ratio observed in severe AD was 0.83 with a standard deviation of 0.48. We conclude that CNS inflammatory associated nucleosomes are typically highly citrullinated.

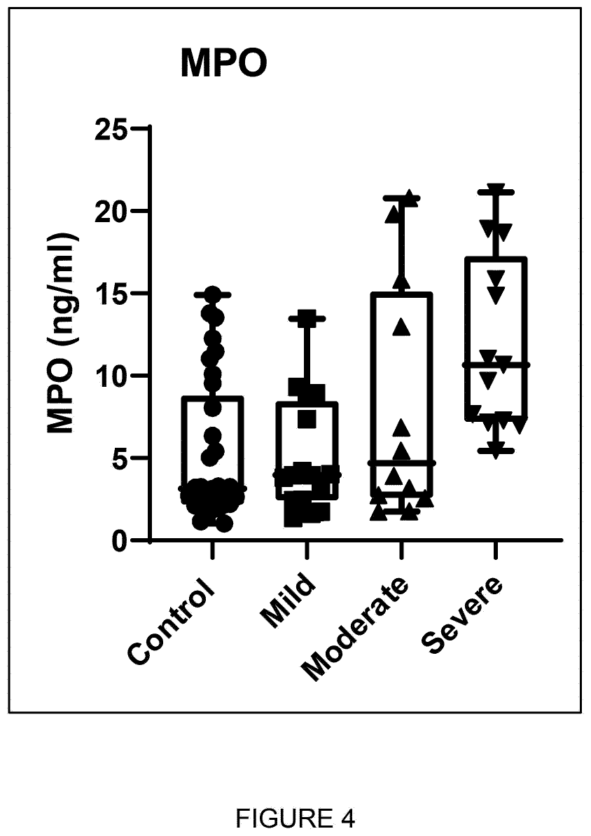

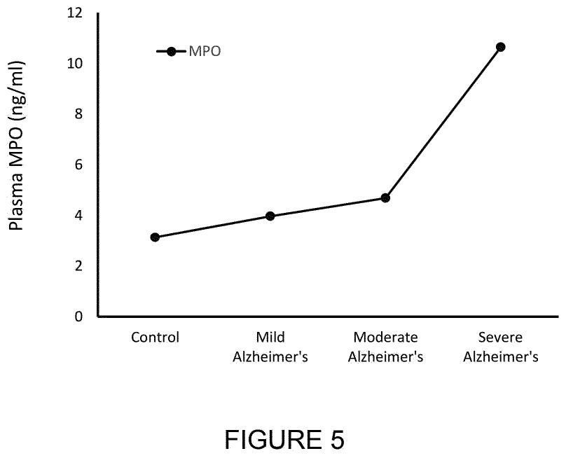

As MPO is a component of NETs, we also measured the plasma MPO in the same patients. We observed disease stage dependent elevated levels of MPO in patients (FIGS. 4 and 5).

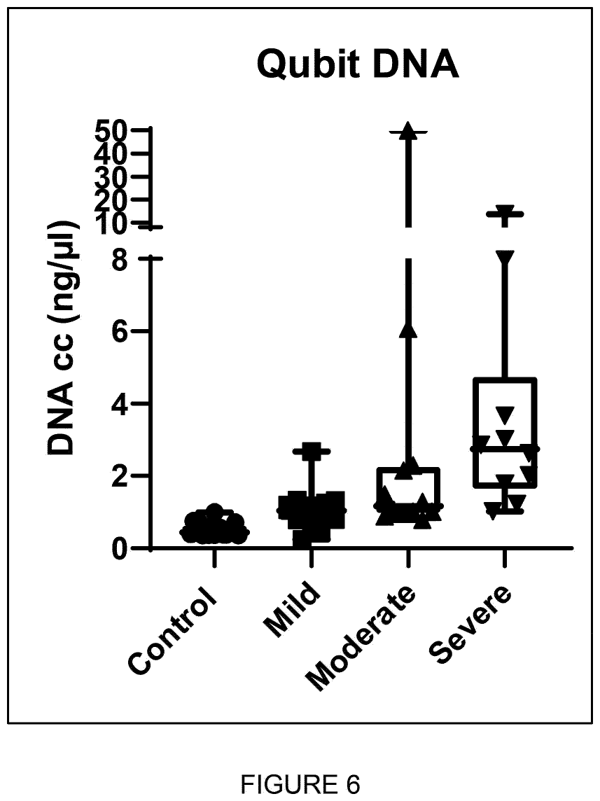

As DNA is a component of NETs and nucleosomes, we also measured the plasma cfDNA levels in the same patients using a DNA dye method and by rQT-PCR. We observed disease stage dependent elevated levels of DNA in patients using both methods (FIGS. 6-8).

We have shown that plasma measurements of NETs, using a variety of parameters for NETs measurements including nucleosomes (H3.1 and H3R8Cit), MPO and cfDNA, are elevated in AD and rise with the severity of the disease. This indicates that NETs measurements may be used to identify subjects with AD and used to monitor the progression of the disease. It also indicates these measurements may be used to monitor the effectiveness of therapy and to help manage the treatment of subjects with dementia, including AD. More generally it indicates that these measurements may be used to monitor the effectiveness of therapy and to help manage the treatment of subject with inflammatory associated disorder of the CNS, such as Parkinson's disease.

We have also shown that plasma measurements of NETs may be used to detect other forms of dementia including those associated with brain injury such as traumatic brain injury (TBI). Testing or monitoring of subjects for TBI may be useful following specific head trauma associated with accidents, for example falling or traffic accidents or concussion. Testing or monitoring of subjects at risk for chronic traumatic encephalopathy (CTE) may be also useful on a regular basis. For example, a periodic test to monitor for cumulative brain damage or CTE among subjects playing contact sports to identify any brain damage early to enable early appropriate action or treatment, before the condition becomes symptomatic later in life.

Methods and uses of the invention may measure the level of (cell free) nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. As described herein, histone proteins form structural units known as nucleosomes which are used to package DNA in eukaryotic cells.

Normal cell turnover in adult humans involves the creation by cell division of a huge number of cells daily and the death of a similar number, mainly by apoptosis. During the process of apoptosis chromatin is broken down into mononucleosomes and oligonucleosomes which are released from the cells. Under normal conditions the levels of circulating nucleosomes found in healthy subjects is reported to be low. Elevated levels are found in subjects with a variety of conditions including many cancers, auto-immune diseases, inflammatory conditions, stroke and myocardial infarction (Holdenrieder & Stieber, Crit. Rev. Clin. Lab. Sci. (2009) 46 (1): 1-24).

The cell free nucleosome may be mononucleosomes, oligonucleosomes, a constituent part of a larger chromatin fragment or a constituent part of a NET or a mixture thereof.

Mononucleosomes and oligonucleosomes can be detected by Enzyme-Linked ImmunoSorbant Assay (ELISA) and several methods have been reported (e.g. (Salgame et al., Nucleic Acids Res. (1997) 25 (3): 680-681; van Nieuwenhuijze et al., Ann. Rheum. Dis. (2003) 62:10-14; Holdenrieder et al. Int. J. Cancer (2001) 95:114-120). These assays typically employ an anti-histone antibody (for example anti-H2B, anti-H3 or anti-H1, H2A, H2B, H3 and H4) as capture antibody and an anti-DNA or anti-H2A-H2B-DNA complex antibody as detection antibody.

Circulating nucleosomes are not a homogeneous group of protein-nucleic acid complexes. Rather, they are a heterogeneous group of chromatin fragments originating from the digestion of chromatin on cell death and include an immense variety of epigenetic structures including particular histone isoforms (or variants), post-translational histone modifications, nucleotides or modified nucleotides, and protein adducts. It will be clear to those skilled in the art that an elevation in nucleosome levels will be associated with elevations in some circulating nucleosome subsets containing particular epigenetic signals including nucleosomes comprising particular histone isoforms (or variants), comprising particular post-translational histone modifications, comprising particular nucleotides or modified nucleotides and comprising particular protein adducts. Assays for these types of chromatin fragments are known in the art (for example, see WO 2005/019826, WO 2013/030579, WO 2013/030578, WO 2013/084002 which are herein incorporated by reference).

The biomarker used in the methods of the invention may be the level of cell free nucleosomes per se and/or an epigenetic feature of a cell free nucleosome. It will be understood that the terms “epigenetic signal structure” and “epigenetic feature” are used interchangeably herein. They refer to particular features of the nucleosome that may be detected. In one embodiment, the epigenetic feature of the nucleosome is selected from the group consisting of: a post-translational histone modification, a histone isoform, a modified nucleotide and/or proteins bound to a nucleosome in a nucleosome-protein adduct. Similarly, the biomarker used in the methods of the invention may be the level of NET or ET per se and/or an epigenetic feature of the NET or ET.

In one embodiment, the epigenetic feature of the nucleosome comprises one or more histone variants or isoforms. The epigenetic feature of the cell free nucleosome may be a histone isoform, such as a histone isoform of a core nucleosome, in particular a histone H3 isoform. The term “histone variant” and “histone isoform” may be used interchangeably herein. The structure of the nucleosome can also vary by the inclusion of alternative histone isoforms or variants which are different gene or splice products and have different amino acid sequences. Many histone isoforms are known in the art. Histone variants can be classed into a number of families which are subdivided into individual types. The nucleotide sequences of a large number of histone variants are known and publicly available for example in the National Human Genome Research Institute NHGRI Histone Database (Mariño-Ramírez et al. The Histone Database: an integrated resource for histones and histone fold-containing proteins. Database Vol. 2011, and http://genome.nhgri.nih.gov/histones/complete.shtml), the GenBank (NIH genetic sequence) Database, the EMBL Nucleotide Sequence Database and the DNA Data Bank of Japan (DDBJ). For example, variants of histone H2 include H2A1, H2A2, mH2A1, mH2A2, H2AX and H2AZ. In another example, histone isoforms of H3 include H3.1, H3.2, H3.3 and H3t. In one embodiment, the histone isoform is H3.1.

The structure of nucleosomes can vary by post translational modification (PTM) of histone proteins. PTM of histone proteins typically occurs on the tails of the core histones and common modifications include acetylation, methylation or ubiquitination of lysine residues as well as methylation or citrullination of arginine residues and phosphorylation of serine residues and many others. Many histone modifications are known in the art and the number is increasing as new modifications are identified (Zhao and Garcia (2015) Cold Spring Harb Perspect Biol, 7: a025064). Therefore, in one embodiment, the epigenetic feature of the cell free nucleosome may be a histone post translational modification (PTM). The histone PTM may be a histone PTM of a core nucleosome, e.g. H3, H2A, H2B or H4, in particular H3, H2A or H2B. In particular, the histone PTM is a histone H3 PTM. Examples of such PTMs are described in WO 2005/019826 (which is herein incorporated by reference).

For example, the post translational modification may include acetylation, methylation, which may be mono-, di- or tri-methylation, phosphorylation, ribosylation, citrullination, ubiquitination, hydroxylation, glycosylation, nitrosylation, glutamination and/or isomerisation (see Ausio (2001) Biochem Cell Bio 79:693). In one embodiment, the histone PTM is selected from citrullination or ribosylation. In a further embodiment, the histone PTM is H3 citrulline (H3cit) or H4 citrulline (H4cit). In a yet further embodiment, the histone PTM is H3cit. In a yet further embodiment, the histone PTM is H3R8cit.

A group or class of related histone post translational modifications (rather than a single modification) may also be detected. A typical example, without limitation, would involve a 2-site immunoassay employing one antibody or other selective binder directed to bind to nucleosomes and one antibody or other selective binder directed to bind the group of histone modifications in question. Examples of such antibodies directed to bind to a group of histone modifications would include, for illustrative purposes without limitation, anti-pan-acetylation antibodies (e.g. a Pan-acetyl H4 antibody [H4panAc]), anti-citrullination antibodies or anti-ubiquitin antibodies.

In one embodiment, the epigenetic feature of the nucleosome comprises one or more DNA modifications. In addition to the epigenetic signalling mediated by nucleosome histone isoform and PTM composition, nucleosomes also differ in their nucleotide and modified nucleotide composition. Some nucleosomes may comprise more 5-methylcytosine residues (or 5-hydroxymethylcytosine residues or other nucleotides or modified nucleotides) than other nucleosomes. In one embodiment, the DNA modification is selected from 5-methylcytosine or 5-hydroxymethylcytosine.

In one embodiment, the epigenetic feature of the nucleosome comprises one or more protein-nucleosome adducts or complexes. A further type of circulating nucleosome subset is nucleosome protein adducts. It has been known for many years that chromatin comprises a large number of non-histone proteins bound to its constituent DNA and/or histones. These chromatin associated proteins are of a wide variety of types and have a variety of functions including transcription factors, transcription enhancement factors, transcription repression factors, histone modifying enzymes, DNA damage repair proteins and many more. These chromatin fragments including nucleosomes and other non-histone chromatin proteins or DNA and other non-histone chromatin proteins are described in the art.

In one embodiment, the protein adducted to the nucleosome (and which therefore may be used as a biomarker) is selected from: a transcription factor, a High Mobility Group Protein or chromatin modifying enzyme. References to “transcription factor” refer to proteins that bind to DNA and regulate gene expression by promoting (i.e. activators) or suppressing (i.e. repressors) transcription. Transcription factors contain one or more DNA-binding domains (DBDs), which attach to specific sequences of DNA adjacent to the genes that they regulate.

All of the circulating nucleosomes and nucleosome moieties, types or subgroups described herein may be useful in the present invention.

It will be understood that more than one epigenetic feature of cell free nucleosomes may be detected in methods and uses of the invention. Multiple biomarkers may be used as a combined biomarker. Therefore, in one embodiment, the use comprises more than one epigenetic feature of cell free nucleosomes as a combined biomarker. The epigenetic features may be the same type (e.g. PTMs, histone isoforms, nucleotides or protein adducts) or different types (e.g. a PTM in combination with a histone isoform). For example, a post-translational histone modification and a histone variant may be detected (i.e. more than one type of epigenetic feature is detected). Alternatively, or additionally, more than one type of post-translational histone modification is detected, or more than one type of histone isoform is detected. In one embodiment of the invention, the use comprises a post-translational histone modification and a histone isoform as a combined biomarker in a sample, for the diagnosis, detection, treatment selection, prognostication or monitoring of an inflammatory condition of the CNS such as AD or other dementia, or Parkinson's disease. In one embodiment, the combined biomarker is H3.1 and H3R8cit.

It will be understood that more than one feature of NETs or ETs may be detected in methods and uses of the invention. Multiple biomarkers may be used as a combined biomarker. Therefore, in one embodiment, the use comprises more than one feature of NETs or ETs as a combined biomarker. The features may be the same type (e.g. PTMs or nucleotides) or different types (e.g. a PTM in combination with a nucleotide sequence).

The term “biomarker” means a distinctive biological or biologically derived indicator of a process, event, or condition. Biomarkers can be used in methods of detection or diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment. Biomarkers are also useful as companion diagnostic products for the selection of patients suitable for treatment by a particular therapy.

The sample may be any biological fluid (also referred to herein as body fluid) sample taken from a subject including, without limitation, cerebrospinal fluid (CSF), whole blood, blood serum, plasma, menstrual blood, endometrial fluid, urine, saliva, or other bodily fluid (stool, tear fluid, synovial fluid, sputum), breath, e.g., as condensed breath, or an extract or purification therefrom, or dilution thereof. In a preferred embodiment, the body fluid sample is selected from blood, serum or plasma. Biological samples also include specimens from a live subject or taken post-mortem. The samples can be prepared, for example where appropriate diluted or concentrated, and stored in the usual manner. It will be understood that methods and uses of the present invention find particular use in blood, serum or plasma samples obtained from a patient.

In one embodiment, the sample is a blood or plasma sample, in particular a plasma sample. In a further embodiment, the sample is a serum sample. In another embodiment, the body fluid sample is a CSF sample.

In one aspect of the invention, there is provided a minimally invasive blood test to detect subjects at high risk for, or to monitor, an inflammatory condition of the CNS in a subject, comprising the measurement of two or more biomarkers in a panel test. In one embodiment the two or biomarkers include a citrullinated nucleosome moiety and another nucleosome moiety to detect the proportion of nucleosomes that are citrullinated in the sample. A high level of citrullination may be used as an indicator of AD or other dementia disorder in the subject.

In one aspect of the invention, there is provided a first minimally invasive front-line test to detect subjects at high risk for AD or other dementia followed by a second test, for subjects identified as high risk, that provides a more definitive and specific test. In one embodiment, the first test is a blood (serum or plasma) test and the second test is a CSF test.

An advantage of a blood test for NETs is that subjects can be tested using a non-invasive or minimally invasive method. One disadvantage of a blood test for NETs is that a positive result (i.e. an elevated level of circulating NETs) cannot be definitively associated with the brain or CNS, but may derive from inflammatory conditions elsewhere in the body.

In contrast, an advantage of a CSF test for NETs is that a positive result is an indicator of CNS derived NETs and one disadvantage of a CSF test for NETs is that the collection of a CSF sample is more difficult for the patient.

Combining a first blood test for NETs, for the identification of subjects at high risk for AD or other dementia, with a second CSF test for NETs, to test for a CNS association of the NETs, combines the advantages of both tests. Subjects in need of a test for dementia but found to be negative for elevated levels of NETs do not undergo an invasive CSF collection. CSF samples are collected from subjects that are identified as being at high risk of AD or other dementia and this test confirms that NETs detected are associated with the CNS.

Therefore, in one embodiment there is a provided a method for the detection or monitoring of AD or other dementia in a subject comprising;
(i) obtaining a blood, serum or plasma sample from the subject;
(ii) measuring the level or amount of NETs in the blood, serum or plasma sample;
(iii) using the level or amount of NETs measured in the blood, serum or plasma sample in step (ii) to identify the subject as being of high or low risk of AD or AD progression;
(iv) obtaining a CSF sample from the subject, if the subject is identified as being of high risk of AD or AD progression in step (iii);
(v) measuring the level or amount of NETs in the CSF sample; and
(vi) using the level or amount of NETs measured in the CSF sample in step (v) as an indicator of inappropriate NETosis of the CNS and AD.

The biomarkers described herein may be used to prepare a kit or panel test, for use in the detection, diagnosis of dementia and in particular of AD and/or monitoring of patients with AD or suspected AD.

Therefore, according to a further aspect of the invention, there is provided a kit comprising reagents to detect one or more nucleosome biomarkers selected from the group consisting of: H3.1 and H3R8cit, for use in diagnosing an inflammatory condition of the CNS, such as AD or other dementia.

According to another aspect of the invention, there is provided the use of a binding agent in the manufacture of a kit for use in a method of diagnosing an inflammatory condition of the CNS, such as AD or other dementia, comprising:
(i) contacting a body fluid sample obtained from the subject with the binding agent to detect or measure the level of cell free nucleosomes or a component thereof; and
(ii) using the level of cell free nucleosomes detected to diagnose said subject as suffering from the condition.

According to another aspect of the invention, there is provided the use of one or more binding agents for detecting one or more nucleosome biomarkers selected from the group consisting of: H3.1 and H3R8cit, in a body fluid sample obtained from a subject in the manufacture of a kit for detecting, diagnosing or monitoring an inflammatory condition of the CNS, such as AD or other dementia.

### Alzheimer's Disease

In one embodiment, methods of the invention may be used to detect, diagnose or monitor Alzheimer's Disease (AD). The most widely used biomarkers for AD measure beta-amyloid, tau, and phospho-tau. These biomarkers are typically measured with positron emission tomography (PET) or by analysis of the patient's cerebrospinal fluid biomarkers. However, PET scans are expensive and often not covered by health insurance companies and the collection of CSF by lumbar puncture is highly invasive. There is therefore a need to develop non-invasive tests for the diagnosing and monitoring of AD. Data is presented herein that shows that use of cell free nucleosomes or components thereof as a biomarker in body fluids was able to discriminate between patients with AD compared to healthy controls.

Mild cognitive impairment (MCI) is an early stage of memory loss or other cognitive ability loss in individuals who maintain the ability to independently perform most activities of daily living. For neurodegenerative diseases, MCI can be an early stage of the disease continuum. Individuals living with MCI who have an abnormal brain PET scan or CSF test for amyloid beta protein are considered to have a diagnosis of MCI due to AD. In one embodiment, the cell free nucleosomes or component thereof is used as a biomarker to differentiate between patients with mild cognitive impairment and patients with AD.

The symptoms of AD can be split into 3 main stages: early-stage AD (mild), middle-stage AD (moderate), and late-stage AD (severe). In one embodiment, the level of cell free nucleosomes or component thereof detected is used as a biomarker to differentiate between patients with early-stage AD (mild), middle-stage AD (moderate), and late-stage AD (severe).

### Dementia

In one embodiment, methods of the invention may be used to detect, diagnose or monitor dementia. As well as AD, neuroinflammation has been associated with forms of dementia including frontotemporal dementia (FTD). However, as with AD, widely used biomarkers for dementia include tau or TDP-43 which are measured using expensive PET scans. The present invention provides inter alia a non-invasive test for the diagnosing and monitoring of dementia.

Dementia is a general term for loss of memory and other mental abilities severe enough to interfere with daily life. It is caused by physical changes in the brain. AD is the most common form of dementia, but the present invention is applicable to other kinds of dementia. Thus, the present invention provides a convenient way to diagnose, monitor and treat inter alia Parkinson's disease, particularly Parkinson's disease dementia, Creutzfeldt-Jakob disease, dementia with Lewy bodies, FTD, including behaviour variant frontotemporal dementia (bvFTD) and primary progressive aphasia (PPA), mixed dementia, posterior cortical atrophy, Parkinson's disease dementia, vascular dementia and Korsakoff syndrome.

### Frailty

In one embodiment, methods of the invention may be used to detect, diagnose or monitor frailty or associated conditions thereof. Frailty is a common condition among older adults. It is associated with a decline in physiological function, increased vulnerability to stresses, and a higher risk of adverse health outcomes. It has been proposed that there is a link between frailty and chronic low-grade inflammation. Understanding the link between frailty and inflammation is important for developing interventions to prevent or manage frailty in older adults. Strategies aimed at reducing chronic inflammation, such as lifestyle modifications, e.g.

regular physical activity, healthy eating, pharmacological interventions, or anti-inflammatory therapies, may hold promise in mitigating the development and progression of frailty. By addressing the underlying inflammatory processes, it may be possible to improve the health and wellbeing of frail individuals, enhancing their quality of life and reducing the burden of age-related frailty.

### Diagnosis and Treatment Methods

References herein to “inflammatory condition of the CNS” refer to a condition in the subject associated with inflammation (or have an inflammation component) of the CNS. Such inflammation may be the result, for example, of a disorder or physical trauma. Therefore, in one embodiment, the inflammatory condition of the CNS is an inflammatory disorder of the CNS. Such disorders include neurodegenerative disorders, such as AD and dementia as described herein. In one embodiment, the methods and uses described herein are for the diagnosis and/or treatment of a neurodegenerative disorder. In one embodiment, the inflammatory disorder of the CNS is an infection or autoimmune disorder.

In an alternative embodiment, the inflammatory condition of the CNS is CNS trauma, such as TBI or CTE.

According to a further aspect of the invention, there is provided a method of detecting, diagnosing or monitoring an inflammatory condition of the CNS in a subject, comprising contacting a body fluid sample obtained from the subject with a binding agent to detect or measure the level of NETs, ETs and/or cell free nucleosomes or a component thereof.

According to a further aspect of the invention, there is provided a method of detecting, diagnosing or monitoring an inflammatory condition of the CNS in a subject, said method comprising obtaining a body fluid sample from the subject; and detecting or measuring the level of NETs, ETs or cell free nucleosomes or a component thereof, to detect, diagnose or monitor the inflammatory condition of the CNS.

According to a further aspect of the invention, there is provided a method of detecting, diagnosing or monitoring an inflammatory condition of the CNS in a subject, said method comprising: (i) measuring or detecting levels of NETs, ETs or cell free nucleosomes or a component thereof in a body fluid sample obtained from the subject, and (ii) comparing the levels determined in (i) to reference levels for NETs, ETs or cell free nucleosomes or a component thereof, respectively, to determine whether the subject is suffering with an inflammatory condition of the CNS or to monitor an inflammatory condition of the CNS in the subject.

According to a further aspect of the invention, there is provided a method for assessing the severity of an inflammatory condition of the CNS in a subject, comprising: (i) measuring levels of NETs, ETs or cell free nucleosomes or a component thereof in a body fluid sample obtained from the subject, and (ii) comparing the levels determined in (i) to reference levels for NETs, ETs or cell free nucleosomes or a component thereof, respectively, to assess the severity of an inflammatory condition of the CNS in the subject.

According to a further aspect of the invention, there is provided a method of monitoring progression of an inflammatory condition of the CNS in a subject previously diagnosed with an inflammatory condition of the CNS, comprising: (i) measuring levels of NETs, ETs or cell free nucleosomes or a component thereof in a body fluid sample obtained from the subject, (ii) comparing the levels determined in (i) to a reference level for NETs, ETs or cell free nucleosomes or a component thereof, respectively, to monitor progression of an inflammatory condition of the CNS in the subject.

According to a further aspect of the invention, there is provided a method of diagnosing AD or other dementia in a patient, comprising:
detecting or measuring the level of NETs, ETs and/or cell free nucleosomes or a component thereof in a body fluid sample obtained from the patient; and
using the level detected in the body fluid sample to determine if the patient has AD or other dementia.

In one embodiment, the method additionally comprises determining at least one clinical parameter for the patient. This parameter can be used in the interpretation of results. Clinical parameters may include any relevant clinical information for example, without limitation, gender, weight, Body Mass Index (BMI), smoking status and dietary habits. Therefore, in one embodiment, the clinical parameter is selected from the group consisting of: family history of dementia, age, sex and body mass index (BMI).

In one embodiment individual assay cut-off levels are used and the patient is considered positive in the panel test if individual panel assay results are above (or below if applicable) the assay cut-off level for all or a minimum number of the panel assays (for example, one of two, two of two, two of three etc). In one embodiment of the invention a decision tree model or algorithm is employed for analysis of the results.

It will be clear to those skilled in the art, that any combination of the nucleosome biomarkers disclosed herein may be used in panels and algorithms for the detection of an inflammatory condition of the CNS and that further markers may be added to a panel including these markers.

In one embodiment, the method described herein is repeated on multiple occasions. This embodiment provides the advantage of allowing the detection results to be monitored over a time period. Such an arrangement will provide the benefit of monitoring or assessing the efficacy of treatment of a disease state. Such monitoring methods of the invention can be used to monitor onset, progression, stabilisation, amelioration, relapse and/or remission. Therefore, in one embodiment the method is repeated on one or more occasions and any changes in the level of cell free nucleosomes or component thereof is used to monitor the progression of an inflammatory condition of the CNS in the patient.

According to a further aspect of the invention, there is provided a method of treating an inflammatory condition of the CNS in a patient, comprising;
(i) detecting or measuring the level of NETs, ETs or cell free nucleosomes or a component thereof in a body fluid sample obtained from the patient;
(ii) using the level of NETs, ETs or cell free nucleosomes or component thereof measured in step (i) as indicative of the presence and/or severity of the inflammatory condition of the CNS in the patient; and
(iii) administering a treatment to the patient if they are determined to have an inflammatory condition of the CNS in step (ii).

According to a further aspect of the invention, there is provided a method of treating AD or other dementia in a patient, comprising;
(i) detecting or measuring the level of cell free nucleosomes or a component thereof in a body fluid sample obtained from the patient;
(ii) using the level of cell free nucleosomes or component thereof measured in step (i) as indicative of the presence and/or severity of dementia in the patient; and
(iii) administering a treatment to the patient if they are determined to have dementia in step (ii).

Treatments available for inflammatory conditions of the CNS, such as AD or other dementia, include pharmacological treatments that target beta-amyloid, such as monoclonal antibodies (e.g., aducanumab), pharmacological treatments for cognitive symptoms, for example cholinesterase inhibitors (e.g., donepezil, rivastigmine, galantamine), glutamate regulators (e.g., memantine), and pharmacological treatments for behavioural and psychological symptoms, for example orexin receptor antagonists (e.g., suvorexant), anxiolytics. antipsychotics and antidepressants. Treatment may also include non-pharmacological treatments such as cognitive rehabilitation.

According to another aspect of the invention there is provided a method of treatment for an inflammatory condition of the CNS, such as AD or other dementia, comprising identifying a patient in need of treatment for the condition by detecting one or more nucleosome biomarkers as described herein, in a body fluid sample obtained from the patient and providing said treatment.

According to another aspect of the invention there is provided a method of treatment for an inflammatory condition of the CNS in a subject comprising:
(i) obtaining a blood, serum or plasma sample from the subject;
(ii) measuring the level or amount of NETs, ETs or cell free nucleosomes or a component thereof in the blood, serum or plasma sample;
(iii) using the level or amount of NETs, ETs or cell free nucleosomes or a component thereof measured in the blood, serum or plasma sample in step (ii) to identify the subject as being of high or low risk of an inflammatory condition of the CNS or progression of an inflammatory condition of the CNS;
(iv) obtaining a CSF sample from the subject, if the subject is identified as being of high risk of an inflammatory condition of the CNS or progression of an inflammatory condition of the CNS in step (iii);
(v) measuring the level or amount of NETs, ETs or cell free nucleosomes or a component thereof in the CSF sample; and
(vi) using the level or amount of NETs, ETs or cell free nucleosomes or a component thereof measured in the CSF sample in step (v) to identify the subject as being in need of treatment for an inflammatory condition of the CNS, and providing said treatment.

According to another aspect of the invention, there is a provided a method of treatment for AD or other dementia in a subject comprising;
(i) obtaining a blood, serum or plasma sample from the subject;
(ii) measuring the level or amount of NETs in the blood, serum or plasma sample;
(iii) using the level or amount of NETs measured in the blood, serum or plasma sample in step (ii) to identify the subject as being of high or low risk of AD or AD progression;
(iv) obtaining a CSF sample from the subject, if the subject is identified as being of high risk of AD or AD progression in step (iii);
(v) measuring the level or amount of NETs in the CSF sample; and
(vi) using the level or amount of NETs measured in the CSF sample in step (v) to identify the subject as being in need of treatment for AD or other dementia, and providing said treatment.

### Additional Biomarkers

The level of NETs, ETs or cell free nucleosomes may be detected or measured as one of a panel of measurements. In one embodiment, the level of NETs, ETs or cell free nucleosomes of component there of detected or measure as using two or more markers.

In one embodiment, the first marker is citrullinated nucleosomes and the second marker is the proportion of nucleosomes that are citrullinated in a sample. In one embodiment, the first marker is histone isoform H3.1 and the second marker is the proportion of nucleosomes that are contain histone isoform H3.1 in a sample.

The panel may comprise different epigenetic features of the nucleosome as described hereinbefore (e.g., a histone isoform and a PTM). Biomarkers useful in a panel test for the detection of AD include, without limitation, amyloid-β (Aβ42), total tau (T-tau), and phosphorylated tau (P-tau).

In one embodiment, the first marker is an epigenetic feature of a cell free nucleosome (such as a histone isoform (e.g. H3.1) or a PTM (e.g. H3R8Cit)) and the second marker is a protein which is adducted to NETs or ETs (such as myeloperoxidase or neutrophil elastase).

Further, additional markers of NETs may be employed. Several proteins occur in NETs that are adducted directly or indirectly to nucleosomes. These proteins include, without limitation, myeloperoxidase (MPO), neutrophil elastase (NE), lactotransferrin, azurocidin, cathepsin G, leukocyte proteinase 3, lysozyme C, neutrophil defensin 1, neutrophil defensin 3, myeloid cell nuclear differentiation antigen, S100 calcium-binding protein A8, S100 calcium-binding protein A9, S100 calcium-binding protein A12, actin β, actin γ, alpha-actin, plastin-2, cytokeratin-10, catalase, alpha-enolase and transketolase (Urban et al., PLOS Pathogens. (2009) 10: e1000639). Any nucleosome-protein adduct that occurs in NETs is a useful adduct for the detection of elevated levels of NETs in methods of the invention. C-reactive protein (CRP) may also be adducted to nucleosomes in NETs and nucleosome-CRP adduct is therefore a useful adduct in methods of the invention.

The panel of measurements may be determined from the same sample, or from different samples. In one embodiment, both markers are obtained from a blood sample. In an alternative embodiment, the first marker is obtained from a blood sample and the second marker is obtained from a different body fluid sample, such as a CSF sample.

### Measurement Methods

In one embodiment, the level of the one or more NETS, ETs or nucleosome biomarkers detected is compared to a control or a reference level. It will be clear to those skilled in the art that the control or reference level may be selected on a variety of basis which may include, for example, the control or reference level may be one of that associated with subjects known to be free of the disease or may be subjects with a different disease (for example, for the investigation of differential diagnosis). The “control” may comprise a healthy subject, a non-diseased subject or a subject without an inflammatory condition of the CNS (such as AD or other dementia) or may be a level determined for an individual earlier in the course of diagnosis. Regarding the latter, the reference level is useful to determine changes in biomarker levels which are indicative of disease progression. Comparison with a control is well known in the field of diagnostics.

It will be understood that it is not necessary to measure healthy/non-diseased controls for comparative purposes on every occasion because once the ‘normal range’ is established it can be used as a benchmark for all subsequent tests. A normal range can be established by obtaining samples from multiple control subjects without an inflammatory condition of the CNS, and testing for the level of biomarker. Results (i.e. biomarker levels) for subjects suspected to have an inflammatory condition of the CNS can then be examined to see if they fall within, or outside of, the respective normal range. Use of a ‘normal range’ is standard practice for the detection of disease.

In one embodiment, the level of NETs, ETs or cell free nucleosomes or component thereof is elevated compared to a control or reference level. In methods in which more than one biomarkers are determined these levels may be compared to a reference wherein the levels or ratios of the plurality of biomarkers in comparison to the reference levels or ratios is indicative that the subject will develop the disorder or that the disorder will progress.

In other words, the levels of the NETs, ETs or cell free nucleosomes is compared to a reference level. By reference level is meant a level of biomarker concentration in a sample against which a test sample is compared.

If a subject is determined to not have dementia or is at a particular stage of disease, then the invention may still be used for the purposes of monitoring any disease progression. For example, if the use comprises a blood, serum or plasma sample from a subject determined not to have dementia, then the biomarker level measurements can be repeated at another time point to establish if the biomarker level has changed.

By stage of disease is meant the severity of the disease classified largely as one of mild (early stage), moderate (mid-stage), or severe (late stage). A determination of stage of disease is important in planning treatment or proposing treatment options.

In one embodiment, there is provided a method for determining the risk of developing AD. The status of a subject may be low, medium or high risk based of calculated levels of biomarkers.

The amounts of the individual biomarkers or patterns of biomarker levels are characteristic of risk state (e.g. low, medium or high). The risk of developing AD is determined by measuring relevant biomarker levels in a subject and comparing the levels with a reference level or pattern of biomarker levels associated with a specific level of risk.

In one embodiment there is provided a method for determining the stage of AD. Each stage is associated with a characteristic amounts of a biomarker (a pattern). The stage of the disease is determined by measuring the levels of the biomarkers and comparing them with reference amounts associated with a particular stage of the disease. For example, biomarker levels can be used to classify between early stage AD and non-AD, or early stage AD and late stage AD. “mild”, “moderate” and “severe” degrees of disease are indicative of increasing level of disease.

In one embodiment, there is provided a method for determining the course of disease in a subject. Disease course refers to changes in disease status over time, whether progressive (worsening) or regressive (improving). Over time, levels in biomarkers may change.

In one embodiment, there is provided a method of monitoring the progression of AD by monitoring the levels of the biomarker over time. For example, the level in a body fluid sample can be collected at a first time (T1) and compared to its level in body fluid sample collected at a second time (T2), in order to determine, quantify or predict the progression of a disorder.

References to “subject” or “patient” are used interchangeably herein. In one embodiment, the patient is a human patient. In one embodiment, the patient is a (non-human) animal. The use, panels and methods described herein are preferably performed in vitro. References to acts carried out on a body fluid sample “obtained” from a subject are intended to encompass acts carried only a body fluid sample already obtained of “obtainable” from a subject and vice versa.

In one embodiment, detection or measurement of one or more of said biomarkers comprises an immunoassay, immunochemical, mass spectroscopy, chromatographic, chromatin immunoprecipitation or biosensor method.

In one embodiment, the method of detection or measurement comprises contacting the body fluid sample with a solid phase comprising a binding agent that detects NETs, ETs or cell free nucleosomes or a component thereof, and detecting binding to said binding agent.

In one embodiment, the detection or measurement comprises an immunoassay. In one embodiment of the invention there is provided a 2-site immunoassay method for nucleosome moieties. In particular, such a method is preferred for the measurement of nucleosomes or nucleosome incorporated epigenetic features in situ employing two anti-nucleosome binding agents or an anti-nucleosome binding agent in combination with an anti-histone modification or anti-histone variant or anti-DNA modification or anti-adducted protein detection binding agent. In another embodiment of the invention, there is provided a 2-site immunoassay employing a labelled anti-nucleosome detection binding agent in combination with an immobilized anti-histone modification or anti-histone variant or anti-DNA modification or anti-adducted protein binding agent.

In one embodiment, the method of detection or measurement comprises: (i) contacting the sample with a first binding agent which binds to an epigenetic feature of NETs, ETs or cell free nucleosomes or a component thereof; (ii) contacting the sample bound by the first binding agent in step (i) with a second binding agent which binds to NETs, ETs or cell free nucleosomes or a component thereof; and (iii) detecting or quantifying the binding of the second binding agent in the sample. In an alternative embodiment, the method of detection or measurement comprises: (i) contacting the sample with a first binding agent which binds to NETs, ETs or cell free nucleosomes or a component thereof; (ii) contacting the sample bound by the first binding agent in step (i) with a second binding agent which binds an epigenetic feature of NETs, ETs or cell free nucleosomes or a component thereof; and (iii) detecting or quantifying the binding of the second binding agent in the sample.

Detecting or measuring the level of the biomarker(s) may be performed using one or more reagents, such as a suitable binding agent. In one embodiment, the one or more binding agents comprises a ligand or binder specific for the desired biomarker, e.g. H3.1 and/or H3R8Cit, or a structural/shape mimic of the biomarker or component part thereof. References to a “biomarker” as used herein may include any single biomarker moiety or a combination of individual biomarker moieties in a biomarker panel.

It will be clear to those skilled in the art that the terms “antibody”, “binder” or “ligand” in regard to any aspect of the invention is not limiting but intended to include any binder capable of binding to particular molecules or entities and that any suitable binder can be used in the method of the invention.

Methods of detecting biomarkers are known in the art. In one embodiment, the reagents comprise one or more ligands or binders. In one embodiment, the ligands or binders of the invention include naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

Diagnostic or monitoring kits (or panels) are provided for performing methods of the invention. Such kits will suitably comprise one or more ligands for detection and/or quantification of the biomarker according to the invention, and/or a biosensor, and/or an array as described herein, optionally together with instructions for use of the kit.

A further aspect of the invention is a kit for detecting the presence of a disease state, comprising a biosensor capable of detecting and/or quantifying one or more of the biomarkers as defined herein. As used herein, the term “biosensor” means anything capable of detecting the presence of the biomarker. Examples of biosensors are described herein. Biosensors may comprise a ligand binder or ligands, as described herein, capable of specific binding to the biomarker. Such biosensors are useful in detecting and/or quantifying a biomarker of the invention.

In one embodiment in which the test is used to derive the progression of the severity of a disorder, the reference substance may be a body fluid sample obtained from the patient at an earlier time. In an additional embodiment, the results of this biomarker testing can be used to prescribe medication. The relative and absolute values of the biomarkers disclosed herein can be used to create a disease profile for an individual, and medication can be selected based upon this profile. Profiling results will allow further monitoring of disease progression and treatment efficacy. In addition the results will permit stratification of patients for clinical drug studies. The subject can then be given medications, which have been shown to provide therapeutic benefit for patients with similar profiles.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace. Biosensors to detect one or more biomarkers of the invention include acoustic, plasmon resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more biomarkers of the invention.

Biomarkers for detecting the presence of a disease are essential targets for discovery of novel targets and drug molecules that retard or halt progression of the disorder. As the result for a biomarker or biomarker panel is indicative of disorder and of drug response, the biomarker is useful for identification of novel therapeutic compounds in in vitro and/or in vivo assays. Biomarkers and biomarker panels of the invention can be employed in methods for screening for compounds that modulate the activity of the biomarker.

Thus, in a further aspect of the invention, there is provided the use of a binder or ligand, as described, which can be a peptide, antibody or fragment thereof or aptamer or oligonucleotide directed to a biomarker according to the invention; or the use of a biosensor, or an array, or a kit according to the invention, to identify a substance capable of promoting and/or of suppressing the generation of the biomarker.

The term “biomarker” means a distinctive biological or biologically derived indicator of a process, event, or condition. Biomarkers can be used in methods of detection, diagnosis, e.g. clinical screening, and prognosis assessment and in monitoring the results of therapy, identifying subjects most likely to respond to a particular therapeutic treatment, drug screening and development. Biomarkers and uses thereof are valuable for identification of new drug treatments and for discovery of new targets for drug treatment.

The term “detecting” or “diagnosing” as used herein encompasses identification, confirmation, and/or characterisation of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

Identifying and/or quantifying can be performed by any method suitable to identify the presence and/or amount of a specific protein in a biological sample from a subject or a purification or extract of a biological sample or a dilution thereof. In methods of the invention, quantifying may be performed by measuring the concentration of the target in the sample or samples. Biological samples that may be tested in a method of the invention include those as defined hereinbefore. The samples can be prepared, for example where appropriate diluted or concentrated, and stored in the usual manner.

Identification and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

For example, detecting and/or quantifying can be performed using an immunological method, such as an immunoassay. Immunoassays include any method employing one or more antibodies or other specific binders directed to bind to the biomarkers defined herein. Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels.

In another example, detecting and/or quantifying can be performed by one or more method(s) selected from the group consisting of: SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving identification and/or quantification of one or more biomarkers of the invention can be performed on bench-top instruments, or can be incorporated onto disposable, diagnostic or monitoring platforms that can be used in a non-laboratory environment, e.g. in the physician's office or at the subject's bedside. Suitable biosensors for performing methods of the invention include “credit” cards with optical or acoustic readers. Biosensors can be configured to allow the data collected to be electronically transmitted to the physician for interpretation and thus can form the basis for e-medicine.

The identification of biomarkers for a disease state permits integration of diagnostic procedures and therapeutic regimes. Detection of a biomarker of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus by monitoring a biomarker of the invention, subject care can be tailored precisely to match the needs determined by the disorder and the pharmacogenomic profile of the subject, the biomarker can thus be used to titrate the optimal dose, predict a positive therapeutic response and identify those subjects at high risk of severe side effects.

Biomarker-based tests provide a first line assessment of ‘new’ subjects, and provide objective measures for accurate and rapid detection and diagnosis, not achievable using the current measures.

Biomarker monitoring methods, biosensors and kits are also vital as subject monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

It will be understood that the embodiments described herein may be applied to all aspects of the invention, i.e. the embodiment described for the uses may equally apply to the claimed methods and so forth.

The invention will now be illustrated with reference to the following non-limiting examples.

### EXAMPLES

### Example 1

EDTA plasma samples were collected from 74 subjects including a cohort of 41 subjects diagnosed with AD and 33 age matched healthy control subjects. Of the 41 subjects with an AD diagnosis, 16 were diagnosed with mild AD, 12 with moderate AD and 13 with severe AD. The plasma samples were analysed for intact cell free nucleosomes containing histone isoform H3.1 using an automated chemiluminescence immunoassay employing an anti-histone H3.1 antibody coated to magnetic particles in combination with a chemiluminescent labelled anti-nucleosome antibody. The results are shown in FIG. 1.

The results show that NETs levels as measured by H3.1 nucleosome levels were elevated in samples taken from patients diagnosed with AD. Moreover, the observed rise in levels correlated with disease severity (FIG. 3). This indicates that the circulating level of nucleosomes containing histone H3.1 may be used to detect or diagnose AD and that sequential testing may be used to monitor the progress of the disease as it becomes more severe and to monitor the efficacy of therapy.

### Example 2

The same 74 EDTA plasma samples were also analysed for intact cell free nucleosomes containing histone H3 citrullinated at arginine amino acid residue 8 (H3R8cit nucleosomes) using an automated chemiluminescence immunoassay employing an anti-histone H3R8cit antibody coated to magnetic particles in combination with a chemiluminescent labelled anti-nucleosome antibody. The results are shown in FIG. 2.

The results show that NETs levels as measured by H3R8cit nucleosome levels were elevated in samples taken from patients diagnosed with AD. As in Example 1, the observed rise in levels correlated with disease severity (FIG. 3). This indicates that the circulating level of citrullinated nucleosomes may be used to detect or diagnose AD and that sequential testing may be used to monitor the progress of the disease as it becomes more severe and to monitor the efficacy of therapy.

### Example 3

The same 74 EDTA plasma samples were also analysed for myeloperoxidase (MPO) using a commercially available MPO ELISA kit. The results are shown in FIG. 4.

The results show that NETs levels as measured by MPO levels were elevated in samples taken from patients diagnosed with AD. The observed rise in MPO levels correlated with disease severity (FIG. 5). This indicates that the circulating level of MPO may be used to detect or diagnose AD and that sequential testing may be used to monitor the progress of the disease as it becomes more severe and to monitor the efficacy of therapy.

### Example 4

The same 74 EDTA plasma samples were also analysed for cell free DNA (cfDNA) using a commercially available Qubit DNA assay. In brief, DNA was extracted from the samples using the QIAamp® DSP Circulating NA kit (Qiagen, #61504) according to manufacturer's protocol. 2 μL of extracted DNA was diluted in 198 μL Qubit working solution and quantified using the Qubit™ 1×dsDNA HS assay Kit (ThermoFisher, #Q32854) following the manufacturer's instructions. The results are shown in FIG. 6.

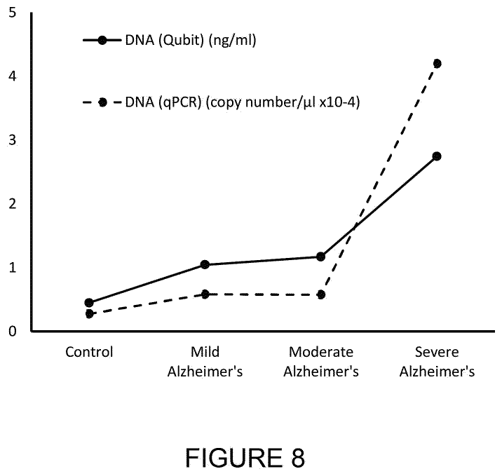

The results show that NETs levels as measured by cfDNA levels were elevated in samples taken from patients diagnosed with AD. The observed rise in levels correlated with disease severity (FIG. 8). This indicates that the circulating cfDNA level may be used to detect or diagnose AD and that sequential cfDNA testing may be used to monitor the progress of the disease as it becomes more severe and to monitor the efficacy of therapy.

### Example 5

The same 74 EDTA plasma samples were also analysed for cell free DNA (cfDNA) using a qRT-PCR assay for the presence of DNA coding for the gene for Hexokinase-2 (qRT-PCR HK2). DNA was extracted from the samples as described in EXAMPLE 4.

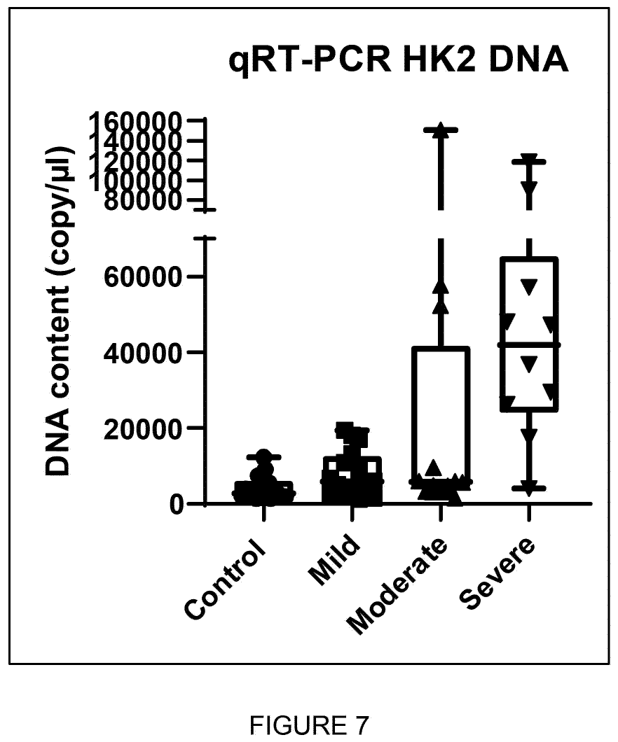

PCR amplification of cfDNA was measured using a real-time quantitative assay for the hexokinase_2 gene. All assays were performed on a CFX96™ Real-Time PCR Detection System (Bio-Rad) using QuantiTect SYBR Green PCR Master Mix (Qiagen, #204143). The primers used were: F, 5′ AACATCGTGTCACCCAGCTA 3′ (SEQ ID NO: 1), and R, 5′ CGTCACAACTGCTAGAAGGC 3′ (SEQ ID NO: 2). PCR was performed at 95 °C for 15 min, followed by 40 cycles of 15 s denaturation at 95 °C, 45 s annealing-extension at 60 °C. The absolute concentration of the target gene was calculated using a standard curve generated using seven different plasmid DNA dilutions containing 1 copy of the Hexokinase-2 gene (2*10-1, 2*10°, 2*101, 2*102, 2*103, 2*104, 2*105, respectively). Each biological replicate was quantified in duplicate, and triplicates of the standard curve were included in each run. The results are shown in FIG. 7.

The results show that NETs levels as measured by cfDNA levels were elevated in samples taken from patients diagnosed with AD.

As in EXAMPLE 4, the observed rise in levels correlated with disease severity (FIG. 8). This indicates that the circulating cfDNA level may be used to detect or diagnose AD and that sequential cfDNA testing may be used to monitor the progress of the disease as it becomes more severe and to monitor the efficacy of therapy.

### Example 6

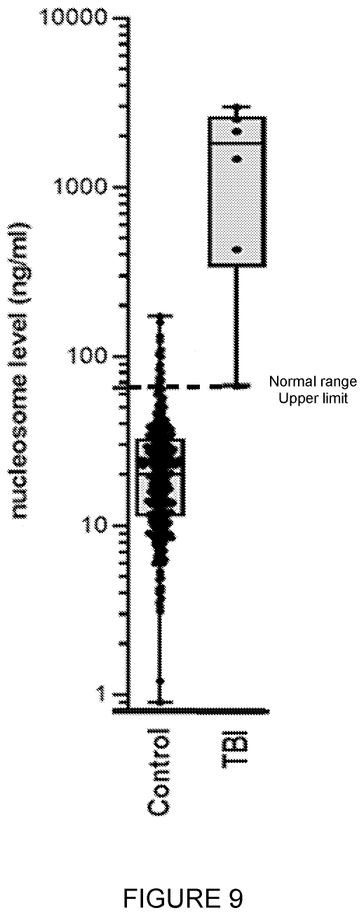

EDTA plasma samples were obtained from 6 subjects diagnosed with a traumatic brain injury (TBI) as well as 269 healthy control subjects. The plasma samples were analysed for intact cell free nucleosomes containing histone isoform H3.1 using an automated chemiluminescence immunoassay employing an anti-histone H3.1 antibody coated to magnetic particles in combination with a chemiluminescent labelled anti-nucleosome antibody. The results are shown in FIG. 9.

The results show that NETs levels as measured by H3.1 nucleosome levels were elevated in samples taken from patients diagnosed with dementias other than AD including those with a physical cause such as TBI. This indicates that the circulating level of nucleosomes containing histone H3.1 may be used to detect or diagnose dementia including that associated with TBI and that sequential testing may be used to monitor the progress of this inflammatory condition of the brain and to monitor the efficacy of therapy.

1-2. (canceled)

3. A method of detecting, diagnosing or monitoring an inflammatory condition of the CNS in a subject, said method comprising: (i) measuring or detecting levels of NETs, ETs or cell free nucleosomes or a component thereof in a body fluid sample obtained from the subject, and (ii) comparing the levels determined in (i) to reference levels for NETs, ETs or cell free nucleosomes or a component thereof, respectively, to determine whether the subject is suffering with an inflammatory condition of the CNS or to monitor an inflammatory condition of the CNS in the subject.

4-5. (canceled)

6. A method of treating an inflammatory condition of the CNS in a patient, comprising;
(i) detecting or measuring the level of NETs, ETs or cell free nucleosomes or a component thereof in a body fluid sample obtained from the patient;
(ii) using the level of NETs, ETs or cell free nucleosomes or component thereof measured in step (i) as indicative of the presence and/or severity of the inflammatory condition of the CNS in the patient; and
(iii) administering a treatment to the patient if they are determined to have an inflammatory condition of the CNS in step (ii).

7. The method of claim 3, wherein the body fluid sample is selected from the group consisting of cerebrospinal fluid, blood, serum and plasma.

8. (canceled)

9. A method of treatment for an inflammatory condition of the CNS in a subject comprising:
(i) obtaining a blood, serum or plasma sample from the subject;
(ii) measuring the level or amount of NETs, ETs or cell free nucleosomes or a component thereof in the blood, serum or plasma sample;
(iii) using the level or amount of NETs, ETs or cell free nucleosomes or a component thereof measured in the blood, serum or plasma sample in step (ii) to identify the subject as being of high or low risk of an inflammatory condition of the CNS or progression of an inflammatory condition of the CNS;
(iv) obtaining a CSF sample from the subject, if the subject is identified as being of high risk of an inflammatory condition of the CNS or progression of an inflammatory condition of the CNS in step (iii);
(v) measuring the level or amount of NETs, ETs or cell free nucleosomes or a component thereof in the CSF sample; and
(vi) using the level or amount of NETs, ETs or cell free nucleosomes or a component thereof measured in the CSF sample in step (v) to identify the subject as being in need of treatment for an inflammatory condition of the CNS, and providing said treatment.

10. (canceled)

11. The method according to claim 3, wherein the inflammatory condition of the CNS is selected from the group consisting of dementia, Alzheimer's disease, and Parkinson's disease.

12-13. (canceled)

14. The method of claim 3, wherein the component comprises an epigenetic feature of the NET, ET or cell free nucleosome.

15. The method of claim 14, wherein the epigenetic feature is a histone isoform or a histone post translational modification (PTM).

16. The method of claim 15, wherein the histone isoform is H3.1.

17. The method of claim 15, wherein the histone post translational modification (PTM) is a histone PTM of a histone H3 or H4.

18. The method of claim 17, wherein the histone PTM is citrullination.

19. The method of claim 18, wherein the histone PTM is selected from H3 citrulline (H3cit) and histone H3 citrullinated at amino acid residue arginine 8 (H3R8cit).

20. The method of claim 3, further comprising determining at least one clinical parameter for the subject.

21-23. (canceled)

24. The method of claim 3, wherein the level of NETs, ETs or cell free nucleosomes of component thereof detected or measured as using two or more markers.

25. The method of claim 24, wherein the first marker is an epigenetic feature of a cell free nucleosome and the second marker is a protein which is adducted to NETs or ETs.

26. The method of claim 24, wherein the first marker is citrullinated nucleosomes and the second marker is the proportion of nucleosomes that are citrullinated in a sample.

27. The method of claim 25, wherein both markers are obtained from a blood sample, or wherein the first marker is obtained from a blood sample and the second marker is obtained from a CSF sample.

28. The method of claim 3, wherein the detecting or measuring is performed using an immunoassay, immunochemical, mass spectroscopy, chromatographic, chromatin immunoprecipitation or biosensor method.

29. The method of claim 3, wherein the method of detection or measurement comprises contacting the body fluid sample with a solid phase comprising a binding agent that detects NETs, ETs or cell free nucleosomes or a component thereof, and detecting binding to said binding agent.

30. The method of claim 3, wherein the method of detection or measurement comprises: (i) contacting the sample with a first binding agent which binds to an epigenetic feature of NETs, ETs or a cell free nucleosome or a component thereof; (ii) contacting the sample bound by the first binding agent in step (i) with a second binding agent which binds to NETs, ETs or cell free nucleosomes or a component thereof; and (iii) detecting or quantifying the binding of the second binding agent in the sample.

31. The method of claim 3, wherein the method of detection or measurement comprises: (i) contacting the sample with a first binding agent which binds to NETs, ETs or cell free nucleosomes or a component thereof; (ii) contacting the sample bound by the first binding agent in step (i) with a second binding agent which binds an epigenetic feature of NETS, ETs or a cell free nucleosome or a component thereof; and (iii) detecting or quantifying the binding of the second binding agent in the sample.

32-42. (canceled)
